(19) United States
(12) Patent Application Publication
(10) Pub. No.: US 2016/0297776 A1
(43) Pub. Date: Oct. 13, 2016

(54) MERCAPTOTRIAZOLE DRAG REDUCER FOR TRANSMISSION PIPELINE AND PREPARATION METHOD THEREFOR

(71) Applicant: PETROCHINA COMPANY LIMITED, Beijing (CN)

(72) Inventors: Guoping LI, Beijing (CN); Zhiheng ZHANG, Beijing (CN); Muyang AI, Beijing (CN); Guangwen LIU, Beijing (CN); Weichun CHANG, Beijing (CN); Haifeng GUO, Beijing (CN); Haihong XU, Beijing (CN); Cheng LIU, Beijing (CN); Shaoyu LI, Beijing (CN)

(73) Assignee: PetroChina Company Limited, Beijing (CN)

(21) Appl. No.: 15/035,214

(22) PCT Filed: Nov. 7, 2014
(86) PCT No.: PCT/CN2014/090570

§ 371 Date: Jul. 1, 2016

(30) Foreign Application Priority Data
Nov. 8, 2013 (CN) 201310551929.9

(51) Int. Cl. C07D 249/04; C09D 7/14; F17D 1/02; C10L 3/00

(52) U.S. Cl. CPC C07D 249/04; C10L 3/003; C09D 7/14; F17D 1/02; C10L 2230/14; C10L 2270/10

(57) ABSTRACT

The present invention provides a mercaptotriazole-based drag-reducing agent for gas pipelines and its preparation method. The drag-reducing agent is prepared by the following steps: producing 1,3-diaminothiourea from hydrazine hydrate and carbon disulfide in a mass ratio of 3:1 to 4:1 under the action of Catalyst I; producing a dithiocarbohydrazone from the condensation reaction of 1,3-diaminothiourea and an aromatic aldehyde in a mass ratio of 1:1 to 1:1.5; producing a mercaptotriazole compound from the dithiocarbohydrazone and an aromatic ester in a mass ratio of 1:1 to 1:3 under the action of Catalyst II; dissolving the mercaptotriazole compound in acetone, adding phosphoric acid or phosphate(s) thereto, and thoroughly mixing them to obtain the target product. The present invention has simple operation and mild reaction conditions and is suitable for online atomization and injection, and the raw materials thereof are available from direct sources.

### TECHNICAL FIELD

The present invention relates to a drag-reducing agent for gas pipelines and the method for preparing the same, and in particular to a mercaptotriazole-based gas pipeline drag-reducing agent for drag reduction in long natural gas pipelines and its preparation method. The present invention pertains to the field of organic macromolecular compounds and preparation methods.

### BACKGROUND ART

Utilization of natural gas as an energy source is now one of the major focuses of domestic and international development. The demand for natural gas has been progressively increasing, thanks to the requirement of sustainable economic development and a craving for “clean energy” worldwide.

Currently, natural gas is transported mainly through pipelines. Modern gas pipelines have been developed for nearly 120 years. When gas flows through pipelines, roughness causes friction, which creates a vortex flow of gas, leading to energy loss and in turn pressure drop along the pipelines. For pipeline transport of natural gas in a turbulent state, roughness of the pipe wall dictates the friction coefficient, and an increase in pipeline throughput requires reduction in roughness of the pipe wall. Recently, tremendous advances have been made in studies on drag reduction in pipeline transport of natural gas. In current research results, drag-reducing methods can be generally categorized into drag-reducing techniques based on coatings lining natural gas pipelines, and drag-reducing techniques using a drag-reducing agent.

It is well recognized that drag-reducing agents for natural gas, like those for crude oil, may significantly increase the pipeline throughput, reduce the power consumption of compressors, lower the installation power of compressors, reduce the number of compressor stations, deliver enormous economic benefits, are much desired in practical production, and have excellent prospects in the market.

However, drag-reducing agents for natural gas are different from drag-reducing agents for commercial liquids (such as petroleum). Drag-reducing agents for liquids, such as that used in the Trans-Alaska pipeline of crude oil, is a typical long-chain polymer having a molecular weight of several million Daltons, which is merged into the liquid phase to reduce vortex flow in the liquid. A drag-reducing agent for liquids expands the bottom layer of the laminar flow from the inner surface of the pipe to the central turbulent area, with its effecting area at the interface between the laminar flow and the turbulent flow. In contrast, drag-reducing agents for natural gas cannot have a very high molecular weight, as their ability to be atomized and ability to “fill” the “pits” in the pipe wall should be considered. Furthermore, drag-reducing agents for natural gas do not take effect at the interface between the laminar flow and the turbulent flow, but directly act on the inner surface of the pipe, where the molecules of the drag-reducing agents are firmly bound to the metal surface to form a smooth, flexible surface, so as to ease off the turbulence at the gas-solid interface and reduce friction between the fluid and the pipe wall, i.e., directly lower the roughness of the inner surface of the pipeline, so that the drag can be reduced without altering the properties of the fluid.

Patent documents U.S. Pat. No. 4,958,653 and U.S. Pat. No. 5,020,561 both propose a method for reducing the drag in a gas pipeline with a drag-reducing agent. The drag-reducing agent is a substance similar to a corrosion inhibitor or lubricant, for example, a fatty acid or an alkoxylated fatty acid amine or amide, having 18 to 54 carbon atoms, the long hydrocarbon chain of which has a molecular weight of about 300 to 900. These inventions further propose that some natural crude oils meet the requirement for a drag-reducing agent. Such natural crude oils consist of asphaltenes, resins and long-chain alkanes (C1-C40), and also comprise a small amount of N, S, O, Fe and V, and these heteroatoms mostly gather in the high-molecular-weight portion, such as the asphaltene portion, to render the asphaltenes polarity.

Although methods for using drag-reducing agents to reduce drag in gas pipelines and drag-reducing compounds have been proposed in the above patent documents, they have a limited drag-reducing effect and are not ideal drag-reducing agents.

### SUMMARY OF INVENTION

The present invention aims to provide a mercaptotriazole-based drag-reducing agent for gas pipelines and its preparation method, which have simple operation and mild reaction conditions and are suitable for online atomization and injection, and the raw materials thereof are available from direct sources.

The drag-reducing agent for natural gas pipelines provided according to the present invention is a mercaptotriazole-based drag-reducing agent for gas pipelines, which is prepared by the following steps:

producing 1,3-diaminothiourea from hydrazine hydrate and carbon disulfide in a mass ratio of 3:1 to 4:1 under the action of Catalyst I; producing a dithiocarbohydrazone from the condensation reaction of 1,3-diaminothiourea and an aromatic aldehyde in a mass ratio of 1:1 to 1:1.5; producing a mercaptotriazole compound from the dithiocarbohydrazone and an aromatic ester in a mass ratio of 1:1 to 1:3 under the action of Catalyst II; dissolving the mercaptotriazole compound in acetone, adding phosphoric acid or phosphate(s) thereto, and thoroughly mixing them to obtain the mercaptotriazole-based drag-reducing agent for gas pipelines.

For the mercaptotriazole-based drag-reducing agent for gas pipelines provided according to the present invention, the Catalyst I used is preferably 2-chloroethanol, 2-mercaptoethanol, ethylene chlorohydrin, or 2-(2-chloroethoxy)ethanol.

For the mercaptotriazole-based drag-reducing agent for gas pipelines provided according to the present invention, the aromatic aldehyde used is preferably benzaldehyde or salicylaldehyde.

For the mercaptotriazole-based drag-reducing agent for gas pipelines provided according to the present invention, the aromatic ester used is preferably ethyl benzoate, n-propyl benzoate, isopropyl benzoate, n-butyl benzoate, isobutyl benzoate, sec-butyl benzoate, t-butyl benzoate, n-pentyl benzoate, isopentyl benzoate, or hexyl benzoate.

For the mercaptotriazole-based drag-reducing agent for gas pipelines provided according to the present invention, the phosphoric acid or phosphate(s) used is preferably one of, or a combination of more of, phosphoric acid, zinc phosphate, sodium phosphate, potassium phosphate, nickel phosphate, ferrous phosphate, and ferric phosphate.

For the mercaptotriazole-based drag-reducing agent for gas pipelines provided according to the present invention, the Catalyst II used is preferably potassium hydroxide or sodium hydroxide.

The present invention further provides a method for preparing the above mercaptotriazole-based drag-reducing agent for gas pipelines, comprising the steps of:

Step 1: producing 1,3-diaminothiourea from hydrazine hydrate and carbon disulfide under the action of Catalyst I;

Step 2: producing a dithiocarbohydrazone from the condensation reaction of 1,3-diaminothiourea and an aromatic aldehyde;

Step 3: producing a mercaptotriazole compound from the dithiocarbohydrazone and an aromatic ester under the action of Catalyst II;

Step 4: dissolving the mercaptotriazole compound in acetone, adding phosphoric acid or phosphate(s) thereto, and thoroughly mixing them to obtain the mercaptotriazole-based drag-reducing agent for gas pipelines.

In the method for preparing the mercaptotriazole-based drag-reducing agent for gas pipelines provided according to the present invention, preferably, Step 1 of producing 1,3-diaminothiourea from hydrazine hydrate and carbon disulfide under the action of Catalyst I comprises the following detailed procedure:

adding dropwise carbon disulfide to a mixed liquid of hydrazine hydrate and ethanol under stirring, wherein the mass ratio of carbon disulfide to hydrazine hydrate is 3:1 to 4:1; allowing them to reflux at 85° C. to 100° C. for 4 to 8 h, followed by cooling in an ice-water bath for 1 to 4 h; performing suction filtration, and adding the filtered product to a solution of Catalyst I dissolved in ethanol to allow reaction at room temperature for 8 to 24 h, followed by refluxing at 95° C. to 105° C. for 4 to 12 h; then adjusting the pH to 2-5 with dilute HCl, followed by suction filtration and oven drying; adding absolute ethanol to allow recrystallization, to obtain 1,3-diaminothiourea.

In the method for preparing the mercaptotriazole-based drag-reducing agent for gas pipelines provided according to the present invention, preferably, Step 2 of producing a dithiocarbohydrazone from the condensation reaction of 1,3-diaminothiourea and an aromatic aldehyde comprises the following detailed procedure:

dissolving the 1,3-diaminothiourea in glacial acetic acid solvent, heating them to boil, and then adding an aromatic aldehyde thereto, wherein the mass ratio of 1,3-diaminothiourea to aromatic aldehyde is 1:1 to 1:1.5; maintaining the boiling state for 4 to 8 h by heating, followed by cooling to room temperature; washing the product obtained by suction filtration with absolute ethanol 3 to 5 times, followed by vacuum drying to obtain the dithiocarbohydrazone.

In the method for preparing the mercaptotriazole-based drag-reducing agent for gas pipelines provided according to the present invention, preferably, Step 3 of producing a mercaptotriazole compound from the dithiocarbohydrazone and an aromatic ester under the action of Catalyst II comprises the following detailed procedure: dissolving the dithiocarbohydrazone in a solution of an aromatic ester in toluene or xylene, wherein the mass ratio of dithiocarbohydrazone to aromatic ester is 1:1 to 1:3; stirring them at a low speed for 12 h at a constant temperature of 80° C. to 100° C.; raising the temperature to 140° C. and performing vacuum distillation to distill off toluene and xylene, so as to obtain the mercaptotriazole compound.

In the method for preparing the mercaptotriazole-based drag-reducing agent for gas pipelines provided according to the present invention, preferably, Step 4 is dissolving the obtained mercaptotriazole compound in acetone, adding phosphoric acid or phosphate(s) thereto, wherein the mass ratio of phosphoric acid/phosphate(s) to mercaptotriazole compound is 3:1 to 3:3, and thoroughly mixing them by stirring, to obtain the target product.

In the method for preparing the mercaptotriazole-based drag-reducing agent for gas pipelines provided according to the present invention, the Catalyst I used is preferably 2-chloroethanol, 2-mercaptoethanol, ethylene chlorohydrin, or 2-(2-chloroethoxy)ethanol.

In the method for preparing the mercaptotriazole-based drag-reducing agent for gas pipelines provided according to the present invention, the aromatic aldehyde used is preferably benzaldehyde or salicylaldehyde.

In the method for preparing the mercaptotriazole-based drag-reducing agent for gas pipelines provided according to the present invention, the aromatic ester used is preferably ethyl benzoate, n-propyl benzoate, isopropyl benzoate, n-butyl benzoate, isobutyl benzoate, sec-butyl benzoate, t-butyl benzoate, n-pentyl benzoate, isopentyl benzoate, or hexyl benzoate.

In the method for preparing the mercaptotriazole-based drag-reducing agent for gas pipelines provided according to the present invention, the phosphoric acid or phosphate(s) used is preferably one of, or a combination of more of, phosphoric acid, zinc phosphate, sodium phosphate, potassium phosphate, nickel phosphate, ferrous phosphate, and ferric phosphate.

In the method for preparing the mercaptotriazole-based drag-reducing agent for gas pipelines provided according to the present invention, the Catalyst II used is preferably potassium hydroxide or sodium hydroxide.

The mercaptotriazole-based drag-reducing agent for gas pipelines according to the present invention employs special macromolecular compounds or polymers having structural characteristics similar to those of surfactants. The polar ends of the macromolecular compounds or polymers firmly bind to the inner metal surface of the pipe to form a smooth film, and the non-polar ends thereof are located at the gas-solid interface formed between the fluid and the inner surface of the pipe. The special molecular structure of the film is used to absorb the turbulence energy at the interface between the fluid and the inner surface to reduce the energy consumed at the inner surface, and the absorbed turbulence energy then dissipates into the fluid to reduce the turbulence disorder, so that the drag is reduced.

The mercaptotriazole-based drag-reducing agent for gas pipelines and the preparation thereof provided according to the present invention have the following advantages:
after introduced into natural gas pipelines, the drag-reducing agent of the present invention adheres to the inner surface of the pipeline and forms a film, thereby significantly reducing the roughness of the inner surface of the pipelines, where the “pits” in the surface are “filled” and the filling is uniform;
in addition, after the film is formed, the corroded products previously existing on the metal surface disappear, indicating the application value of the drag-reducing agent of the present invention in drag reduction; the film on the surface has good flexibility, indicating that the synthetic pyridine salt-based drag-reducing agent possesses properties required by drag-reducing agents for natural gas, and its film-forming performance and flexibility amply demonstrate its potential application value as a drag-reducing agent for natural gas pipelines;
the drag-reducing agent according to the present invention shows a remarkable drag-reducing effect in gas pipelines; its raw materials are available from direct sources, and its preparation method has simple operation, mild reaction conditions, and low requirements on instruments, and easily realizes large-scale industrial production.

### DESCRIPTION OF DRAWINGS

FIG. 1 is a SEM image of the original surface of a steel sheet;

FIG. 2 is a SEM image of the surface of the steel sheet coated with the perfluorophosphate-based drag-reducing agent for gas pipelines according to Example 3.

### DETAILED DESCRIPTION OF INVENTION

In order to allow better understanding of the technical features, objectives and beneficial effects of the present invention, detailed description of the technical solutions of the present invention will be provided below, but should not to be construed as limiting the scope of the present invention.

### Example 1

This example provides a mercaptotriazole-based drag-reducing agent for gas pipelines and its preparation method, the method specifically comprising the following steps:
adding dropwise 100 g carbon disulfide to a mixed liquid of 25 g hydrazine hydrate and 50 g ethanol under stirring; allowing them to reflux at 100° C. for 8 h, followed by cooling in an ice-water bath for 4 h; performing suction filtration, and adding the filtered product to a solution of 2-chloroethanol dissolved in ethanol to allow reaction at room temperature for 24 h, followed by refluxing at 100° C. for 12 h; then adjusting the pH to 5 with dilute HCl, followed by suction filtration and oven drying; adding absolute ethanol to allow recrystallization, to obtain 197 g 1,3-diaminothiourea;
dissolving the 1,3-diaminothiourea in glacial acetic acid solvent, heating them to boil, then adding 200 g aromatic aldehyde thereto, and maintaining the boiling state for 8 h by heating, followed by cooling to room temperature; washing the product obtained by suction filtration with absolute ethanol 5 times, followed by vacuum drying to obtain 311 g dithiocarbohydrazone;
dissolving the dithiocarbohydrazone in a solution of 300 g aromatic ester in toluene or xylene; stirring them at a low speed for 12 h at a constant temperature of 100° C.; raising the temperature to 140° C. and performing vacuum distillation to distill off toluene and xylene, so as to obtain a mercaptotriazole compound;
dissolving the mercaptotriazole compound in 800 ml acetone, adding 300 g phosphoric acid thereto, and thoroughly mixing them by stirring, to obtain the target product.

### Example 2

This example provides a mercaptotriazole-based drag-reducing agent for gas pipelines and its preparation method, the method specifically comprising the following steps:
adding dropwise 100 g carbon disulfide to a mixed liquid of 30 g hydrazine hydrate and 60 g ethanol under stirring; allowing them to reflux at 100° C. for 7 h, followed by cooling in an ice-water bath for 2 h; performing suction filtration, and adding the filtered product to a solution of 2-chloroethanol dissolved in ethanol to allow reaction at room temperature for 24 h, followed by refluxing at 100° C. for 11 h; then adjusting the pH to 3 with dilute HCl, followed by suction filtration and oven drying; adding absolute ethanol to allow recrystallization, to obtain 213 g 1,3-diaminothiourea;
dissolving the 1,3-diaminothiourea in glacial acetic acid solvent, heating them to boil, then adding 220 g aromatic aldehyde thereto, and maintaining the boiling state for 8 h by heating, followed by cooling to room temperature; washing the product obtained by suction filtration with absolute ethanol 3 times, followed by vacuum drying to obtain 342 g dithiocarbohydrazone;
dissolving the dithiocarbohydrazone in a solution of 350 g aromatic ester in toluene or xylene; stirring them at a low speed for 10 h at a constant temperature of 80° C.; raising the temperature to 140° C. and performing vacuum distillation to distill off toluene and xylene, so as to obtain a mercaptotriazole compound;
dissolving the mercaptotriazole compound in 800 ml acetone, adding 350 g phosphoric acid thereto, and thoroughly mixing them by stirring, to obtain the target product.

### Example 3

This example provides a mercaptotriazole-based drag-reducing agent for gas pipelines and its preparation method, the method specifically comprising the following steps:
adding dropwise 100 g carbon disulfide to a mixed liquid of 28 g hydrazine hydrate and 55 g ethanol under stirring; allowing them to reflux at 100° C. for 8 h, followed by cooling in an ice-water bath for 3 h; performing suction filtration, and adding the filtered product to a solution of 2-chloroethanol dissolved in ethanol to allow reaction at room temperature for 20 h, followed by refluxing at 100° C. for 12 h; then adjusting the pH to 4 with dilute HCl, followed by suction filtration and oven drying; adding absolute ethanol to allow recrystallization, to obtain 204 g 1,3-diaminothiourea;
dissolving the 1,3-diaminothiourea in glacial acetic acid solvent, heating them to boil, then adding 200 g aromatic aldehyde thereto, and maintaining the boiling state for 8 h by heating, followed by cooling to room temperature; washing the product obtained by suction filtration with absolute ethanol 5 times, followed by vacuum drying to obtain 319 g dithiocarbohydrazone;
dissolving the dithiocarbohydrazone in a solution of 320 g aromatic ester in toluene or xylene; stirring them at a low speed for 12 h at a constant temperature of 100° C.; raising the temperature to 140° C. and performing vacuum distillation to distill off toluene and xylene, so as to obtain a mercaptotriazole compound;
dissolving the mercaptotriazole compound in 700 ml acetone, adding 330 g phosphoric acid thereto, and thoroughly mixing them by stirring, to obtain the target product.

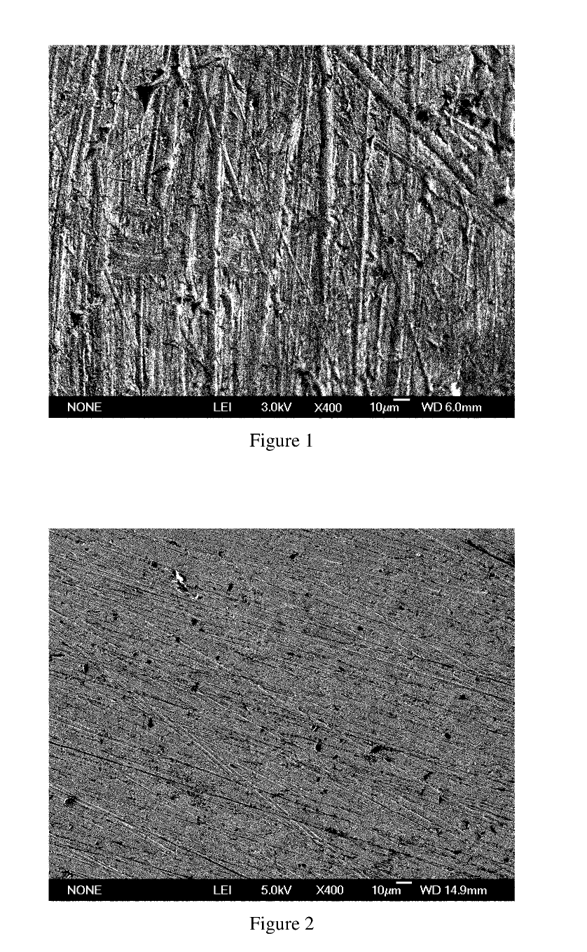

The mercaptotriazole-based drag-reducing agent for gas pipelines obtained according to this example was subjected to a film-forming process and an electronic microscopic analysis by dissolving a certain amount of the product in an appropriate amount of solvent (such as diesel, kerosene, butanol, hexanol, octanol, etc), immersing a treated (degreased, rust removed, and polished with metallographic sand paper) steel sheet (the SEM image of the original surface of the sheet is shown in FIG. 1) in the solvent, taking the sheet out after a certain period of time and drying it, and subjecting it to a microscopic analysis on a JEDL JSM-6700F scanning electronic microscope.

The results show that for the steel sheet surface coated with the mercaptotriazole-based drag-reducing agent for gas pipelines according to this example (as shown in FIG. 2), its roughness has been significantly improved, wherein the “pits” in the surface are “filled” and the filling is uniform. Furthermore, after a film was formed, the mercaptotriazole-based drag-reducing agent for gas pipelines was tested in the in-door evaluation system for natural gas drag-reducing agents (according to the indoor testing method for drag-reducing effects of drag-reducing agents for natural gas, Standard No.: Q/SY GD0221-2012). The results show that the drag reduction percentage is greater than 18%, and this effect stably persists for over 60 days, indicating that the mercaptotriazole-based drag-reducing agent for gas pipelines is valuable in drag reduction applications for gas pipelines.

1. A mercaptotriazole-based drag-reducing agent for gas pipelines, which is prepared by the following steps:
producing 1,3-diaminothiourea from hydrazine hydrate and carbon disulfide in a mass ratio of 3:1 to 4:1 under the action of a Catalyst I;
producing a dithiocarbohydrazone from the condensation reaction of 1,3-diaminothiourea and an aromatic aldehyde in a mass ratio of 1:1 to 1:1.5;
producing a mercaptotriazole compound from the dithiocarbohydrazone and an aromatic ester in a mass ratio of 1:1 to 1:3 under the action of a Catalyst II; and
dissolving the mercaptotriazole compound in acetone, adding phosphoric acid or phosphate(s) thereto, and thoroughly mixing them to obtain the mercaptotriazole-based drag-reducing agent for gas pipelines.

2. The mercaptotriazole-based drag-reducing agent for gas pipelines according to claim 1, wherein the Catalyst I is 2-chloroethanol, 2-mercaptoethanol, ethylene chlorohydrin, or 2-(2-chloroethoxy)ethanol.

3. The mercaptotriazole-based drag-reducing agent for gas pipelines according to claim 1, wherein the aromatic aldehyde is benzaldehyde or salicylaldehyde.

4. The mercaptotriazole-based drag-reducing agent for gas pipelines according to claim 1, wherein the aromatic ester is ethyl benzoate, n-propyl benzoate, isopropyl benzoate, n-butyl benzoate, isobutyl benzoate, sec-butyl benzoate, t-butyl benzoate, n-pentyl benzoate, isopentyl benzoate, or hexyl benzoate.

5. The mercaptotriazole-based drag-reducing agent for gas pipelines according to claim 1, wherein the phosphoric acid or phosphate(s) is selected from the group consisting of one of, or a combination of more than one of, phosphoric acid, zinc phosphate, sodium phosphate, potassium phosphate, nickel phosphate, ferrous phosphate and ferric phosphate.

6. The mercaptotriazole-based drag-reducing agent for gas pipelines according to claim 1, wherein the Catalyst II is potassium hydroxide or sodium hydroxide.

7. A method for preparing the mercaptotriazole-based drag-reducing agent for gas pipelines of claim 1, comprising the steps of:
Step 1: producing 1,3-diaminothiourea from hydrazine hydrate and carbon disulfide under the action of Catalyst I;
Step 2: producing a dithiocarbohydrazone from the condensation reaction of 1,3-diaminothiourea and an aromatic aldehyde;
Step 3: producing a mercaptotriazole compound from the dithiocarbohydrazone and an aromatic ester under the action of Catalyst II; and
Step 4: dissolving the mercaptotriazole compound in acetone, adding phosphoric acid or phosphate(s) thereto, and thoroughly mixing them to obtain the mercaptotriazole-based drag-reducing agent for gas pipelines.

8. The method for preparing the mercaptotriazole-based drag-reducing agent for gas pipelines according to claim 7, wherein Step 1 of producing 1,3-diaminothiourea from hydrazine hydrate and carbon disulfide under the action of Catalyst I comprises the following procedure:
adding dropwise carbon disulfide to a mixed liquid of hydrazine hydrate and ethanol, wherein the mass ratio of carbon disulfide to hydrazine hydrate is 3:1 to 4:1;
allowing them to reflux at 85° C. to 100° C. for 4 to 8 h, followed by cooling in an ice-water bath for 1 to 4 h;
performing suction filtration, and adding the filtered product to a solution of Catalyst I dissolved in ethanol to allow reaction at room temperature for 8 to 24 h, followed by refluxing at 95° C. to 105° C. for 4 to 12 h; and
then adjusting the pH to 2-5 with dilute HCl, followed by suction filtration and oven drying; adding absolute ethanol to allow recrystallization, to obtain 1,3-diaminothiourea.

9. The method for preparing the mercaptotriazole-based drag-reducing agent for gas pipelines according to claim 7, wherein Step 2 of producing a dithiocarbohydrazone from the condensation reaction of 1,3-diaminothiourea and an aromatic aldehyde comprises the following procedure:
dissolving the 1,3-diaminothiourea in glacial acetic acid, heating them to boil, and then adding an aromatic aldehyde thereto, wherein the mass ratio of 1,3-diaminothiourea to aromatic aldehyde is 1:1 to 1:1.5;
maintaining the boiling state for 4 to 8 h by heating, followed by cooling to room temperature; and
washing the product obtained by suction filtration with absolute ethanol 3 to 5 times, followed by vacuum drying to obtain the dithiocarbohydrazone.

10. The method for preparing the mercaptotriazole-based drag-reducing agent for gas pipelines according to claim 7, wherein Step 3 of producing a mercaptotriazole compound from the dithiocarbohydrazone and an aromatic ester under the action of Catalyst II comprises the following procedure:
dissolving the dithiocarbohydrazone in a solution of an aromatic ester in toluene or xylene, wherein the mass ratio of dithiocarbohydrazone to aromatic ester is 1:1 to 1:3;
stirring them at a low speed for 12 h at a constant temperature of 80° C. to 100° C.; and
raising the temperature to 140° C. and performing vacuum distillation to distill off toluene and xylene, so as to obtain the mercaptotriazole compound.

11. The method for preparing the mercaptotriazole-based drag-reducing agent for gas pipelines according to claim 7, wherein Step 4 is dissolving the obtained mercaptotriazole compound in acetone, adding phosphoric acid or phosphate(s) thereto, wherein the mass ratio of phosphoric acid/phosphate(s) to mercaptotriazole compound is 3:1 to 3:3, and thoroughly mixing them by stirring, to obtain the target product.

12. The method for preparing the mercaptotriazole-based drag-reducing agent for gas pipelines according to claim 7, wherein the Catalyst I is 2-chloroethanol, 2-mercaptoethanol, ethylene chlorohydrin, or 2-(2-chloroethoxy)ethanol.

13. The method for preparing the mercaptotriazole-based drag-reducing agent for gas pipelines according to claim 7, wherein the aromatic aldehyde is benzaldehyde or salicylaldehyde.

14. The method for preparing the mercaptotriazole-based drag-reducing agent for gas pipelines according to claim 7, wherein the aromatic ester is ethyl benzoate, n-propyl benzoate, isopropyl benzoate, n-butyl benzoate, isobutyl benzoate, sec-butyl benzoate, t-butyl benzoate, n-pentyl benzoate, isopentyl benzoate, or hexyl benzoate.

15. The method for preparing the mercaptotriazole-based drag-reducing agent for gas pipelines according to claim 7, wherein the phosphoric acid or phosphate(s) is selected from the group consisting of one of, or a combination of more than one of, phosphoric acid, zinc phosphate, sodium phosphate, potassium phosphate, nickel phosphate, ferrous phosphate and ferric phosphate.

16. The method for preparing the mercaptotriazole-based drag-reducing agent for gas pipelines according to claim 7, wherein the Catalyst II is potassium hydroxide or sodium hydroxide.
